(19) United States
(12) Patent Application Publication
(10) Pub. No.: US 2022/0273862 A1
(43) Pub. Date: Sep. 1, 2022

(54) DEVICE AND PROCESS FOR RECEIVING SUCTIONED SECRETION, INCLUDING A BACKFLOW BARRIER

(71) Applicant: Drägerwerk AG & Co. KGaA, Lübeck (DE)

(72) Inventors: Heiko LOKOTSCH, Lübeck (DE); Thomas REINBOTH, Lübeck (DE); Benjamin FLEITMANN, Lübeck (DE)

(21) Appl. No.: 17/632,657

(22) PCT Filed: Jul. 14, 2020
(86) PCT No.: PCT/EP2020/069817

§ 371 Date: Feb. 3, 2022

(30) Foreign Application Priority Data
Aug. 7, 2019 (DE) 10 2019 005 532.2

(51) Int. Cl. A61M 1/00

(52) U.S. Cl. CPC A61M 1/784; A61M 1/604; A61M 1/732

(57) ABSTRACT

A collection device and a collection process provide the capability of receiving secretions, which have been suctioned out of a patient (P). A collecting unit including a bag (3) and a cover (2) encloses a collecting unit interior space (In1). The suctioned secretions flow through a patient-side fluid connection and through a patient-side port (4) in the cover into the collecting unit interior space. A backflow barrier includes a blocking body, which is fastened to a surface of the cover of the collecting unit interior space. In a blocking state, the blocking body closes the patient-side port. A restoring force generator exerts a restoring force on the blocking body, to hold the backflow barrier in the blocking state. A flow of secretions through the patient-side port into the collecting unit interior space brings about a transference of the backflow barrier into a flow stat e against the restoring force.

### CROSS REFERENCE TO RELATED APPLICATIONS

This application is a United States National Phase Application of International Application PCT/EP2020/069817, filed Jul. 14, 2020, and claims the benefit of priority under 35 U.S.C. § 119 of German Application 10 2019 005 532.2, filed Aug. 7, 2019, the entire contents of which are incorporated herein by reference.

### TECHNICAL FIELD

The present invention pertains to a collection device and to a collection process, which are capable of receiving secretions (secretion) that have been suctioned out of a patient, wherein a backflow barrier prevents or at least reduces the backflow of received secretions.

### TECHNICAL BACKGROUND

Various such devices have become known.

A collection device (suction device 3100), which is capable of receiving a fluid in a container (container 3300) for holding the drained fluid), is described in the embodiment according to FIG. 5 from US 2007/0027433 A1. The fluid flows from the patient through a drain 3220, a drain tube 3210 and a patient-side port (input passage 3200) into the container 3300. A valve (reversible valve 3700), which comprises a nonreturn valve (one-way valve 3400) with a sealing element (seal 3410) and is inserted into a cover of the container 3300, is arranged between the patient-side port 3200 and the container 3300. A flow of fluid through the patient-side port 3200 into the container 3300 forces the element 3410 into an open position. A flow out of the container 3300 in the direction of the patient-side port 320 closes the element 3410. The nonreturn valve 3400 comprises a Heimlich valve in one embodiment.

The embodiment according to FIG. 3 from US 2018/0318474 A1 shows a container (collection canister 232), which has a bag (collection bag 202) in its interior (chamber 204a) and is closed by a closure (cover 206). A patient-side port (inlet port 206a) and a device-side port (outlet port 206b) are arranged in the closure 206. Fluid flows out of a wound of a patient through a wound dressing (wound dressing 112), a coupling unit (port or connector 107), a line (conduit 106) and the patient-side port 206a to the bag 202. The device-side port 206b can be connected to a vacuum source (suction pump 118). A nonreturn valve (check valve 212) prevents fluid from flowing back out of the bag 202 again. This nonreturn valve 212 may be arranged in the closure 206.

In WO 95/24230 A1, a container 1 can be closed by a cover 2 in a vacuum-tight manner. A hose 4 is attached to a bag 3, which is capable of receiving secretions and is inserted in the container 1. The hose 4 is led outwards out of the interior of the container 1 through an opening 5 by means of a sealing element 6. The opening 5 may be located in the upper part of the container wall or even in the cover 2. When the cover 2 is closed, the sealing element 6 closes the opening 5 in a vacuum-tight manner. The sealing element 6 is inserted into the opening 5. The filled bag 3 can be removed by the cover 2 being opened and the sealing element 6 together with the hose 4 being pulled out of the opening 5. The bag 3 is disposed of together with the hose 4 and with the sealing element 6.

EP 1984043 B1 shows in FIG. 3 an outer bag (suction canister 320), into which a disposable bag (suction bag 300), which is fastened to a cover (cover 301), is fastened. A vacuum generator (underpressure apparatus 340) is fastened to a connector (underpressure connector 322) at the outer container 320. Different ways of how the disposable bag 300 can be fastened to the outer bag 320 (fastening means 330, 332) are mentioned. As examples, adhesive tape, adhesive and a mechanical connection are mentioned. In one embodiment, the fastening means 330, 332 binds the disposable bag 300, and the vacuum opens the fastening means 3320, 332 and brings about that the disposable bag 300 is arranged correctly in the outer container 320. It is also possible that the fastening means 330, 332 is opened by the secretions, which flow into the disposable bag 300.

The container 112 of WO 2017/132199 A1 comprises a canister 154 and a cover (lid 310), wherein the cover 310 may have a snap closure 312 for a projection 160 at the canister 154. A first vacuum source 104 can be connected to a port 180 arranged downstream. A connection to a patient-side connection (dressing 102) can be established at a port 182 arranged upstream. In one embodiment, a second vacuum source, for example, a reservoir 300 with a membrane 320, is integrated into the cover 310, wherein the reservoir 300 provides a vacuum chamber 304. The membrane 320 is sufficiently flexible and impermeable to fluid. “Medical grade silicone” is mentioned as a possible material for the membrane 320. The connected first vacuum source 104 reduces the pressure in the vacuum chamber 304. The pressure in the vacuum chamber 304 in relation to the collection area 170 is reduced as a result. Thanks to the vacuum, the flexible membrane 420 compresses a plurality of springs 324 at the cover 310. The compressed vacuum chamber 304 may have a pressure, which is equal to the ambient pressure. If the first vacuum source 104 is separated from the connection 180, a valve (check valve 314) seals the vacuum chamber 304. The springs 324 expand and stretch the flexible membrane 320. As a result, the volume of the vacuum chamber 304 is increased. The reservoir 300 may maintain a vacuum in relation to the patient-side connection 102.

A system 1 for collecting fluid, which comprises a main container 2 with a main collection chamber 10 and a first secondary container 3 with a secondary collection chamber 20, is described in DE 10 2017 007 701 A1. The three main channel sections 11, 12, 13 are in fluid communication with the main collection chamber 10 and comprise each a water-soluble closing element 14, 15, 16 as well as optionally a respective blocking element 17, 18, 19, wherein the elements 14 through 19 may be configured as self-closing membranes. The first secondary channel section 21 can be inserted into the first main channel section 11, wherein the first blocking element 17 is pierced.

A device for removing secretions from the respiratory tract of a patient is described in DE 1038717. A breathing mask or a mouthpiece is pulled over the mouth and/or the nose of the patient and is connected to a vacuum source by means of a valve. An artificial coughing fit, which pulls secretions out of the patient, is achieved by the valve being actuated briefly. The breathing mask or the mouthpiece is in connection with the outside air via an additional valve, so that the patient is able to inhale and exhale. A control unit closes the additional valve and opens the valve when a suctioning shall be carried out.

Collection devices are, in addition, described in US 2015/0141943 A1, US 2011/0067699 A1, US 2013/0341330 A1 and in WO 2008/144951 A1.

### SUMMARY

An underlying object of the present invention is to provide a collection device and a process for receiving secretions that have been suctioned out of a patient, which device and process are capable of receiving suctioned secretions in a collecting unit and of reducing the backflow of secretions in a mechanically simpler manner than prior-art collection devices.

The object is accomplished by a collection device with the features according to the invention as well as by a process with the features according to the invention. Advantageous embodiments are described. Advantageous embodiments of the collection device according to the present invention are, insofar as meaningful, also advantageous embodiments of the process according to the present invention and vice versa.

The collection device according to the present invention is capable of receiving secretions, wherein such secretions are being suctioned or has been suctioned out of a patient.

The collection device comprises a collecting unit. This collecting unit comprises a closure and a bag. The closure is especially a cover that is preferably located above the bag during the operation. The bag is preferably expandable. The collecting unit encloses a collecting unit interior space, and especially in a preferably fluid-tight manner. The collecting unit is capable of receiving secretions in this collecting unit interior space, which secretions are being suctioned or has been suctioned out of a patient.

The collection device comprises, furthermore, a patient-side port. This patient-side port makes it possible to establish a patient-side fluid connection between a patient-side coupling unit located outside the collecting unit and the collecting unit interior space. Secretions can be delivered into the collecting unit interior space through this patient-side fluid connection.

The collection device further comprises a backflow barrier for the patient-side port. This backflow barrier comprises a blocking body and a restoring force generator. The blocking body is fastened to a surface of the closure, especially to a surface, which faces towards the collecting unit interior space, i.e., on the inner side of the closure.

The backflow barrier can be transferred into a passing (flow) state and into a blocking state. At least when the backflow barrier is in the passing state, secretions can flow through the patient-side port into the collecting unit interior space. When the backflow barrier is in the blocking state, then the blocking body completely or at least partially blocks the patient-side port. As a result, the blocking body prevents a flow of secretions out of the collecting unit interior space and into the patient-side port. The blocking body at least reduces this flow, i.e., the flow rate of this flow, into the patient-side port compared with the passing state.

The restoring force generator exerts a restoring force on the blocking body. This restoring force aims to transfer the backflow barrier into the blocking state and to hold it in the blocking state.

A flow of secretions through the patient-side port into the collecting unit interior space brings about that the backflow barrier is transferred against the exerted restoring force into the passing state. As soon as the flow rate of this flow falls below a threshold due to the construction of the backflow barrier, the backflow barrier is transferred into the blocking state by the restoring force generator.

The process according to the present invention for receiving secretions by means of such a collection device comprises the following steps:
The restoring force generator exerts a restoring force, which holds the backflow barrier in the blocking state.
The patient-side port is connected to a patient-side coupling. As a result, a patient-side fluid connection is established between the patient-side coupling unit and the collecting unit interior space. This patient-side fluid connection leads through the patient-side port.
A flow of secretions from the patient-side coupling unit through the patient-side fluid connection into the collecting unit is brought about.
The backflow barrier is transferred into the passing state by the brought-about flow of secretions against the restoring force. As a result, the secretions can flow into the collecting unit interior space.
Subsequently, the flow of secretions into the collecting unit is terminated. The restoring force again transfers the backflow barrier into the blocking state.
In the blocking state, the blocking body completely or at least partially closes the patient-side port. As a result, the flow of secretions is reduced or even inhibited.

The collecting unit is typically connected to the patient-side coupling unit, receives suctioned secretions and is then removed from the location of use. As a rule, it is disposed of together with the secretions. The backflow barrier according to the present invention is held by the nonrestoring force generator in the blocking state and therefore prevents suctioned secretions from again being released from the collecting unit. After the suctioning is completed, the release of the secretions may occur without a backflow barrier especially if there is no longer a fluid connection. A component of the collecting unit, especially an elastic bag, is, as a rule, expanded and aims to contract again. Without a suitable protection mechanism, the expanded or dropped collecting unit could therefore discharge suctioned secretions through the patient-side port into the environment. This is undesirable.

It is possible, after completion of the suctioning, to close the patient-side port by means of a cap or another closing element. However, the situation may occur that such a closing element is not at all placed onto the patient-side port or is not placed correctly or too late onto the patient-side port and secretions are therefore discharged in spite of such a closing element. This may happen especially if the collecting unit is exposed to a mechanical pulse from outside, for example, it falls onto the floor. The backflow barrier according to the present invention also prevents in this situation secretions from being discharged, or at least significantly reduces the quantity of secretions discharged. The collection device may have the backflow barrier according to the present invention and additionally a closing element, or the collection device is equipped without such a closing element.

According to the present invention, the backflow barrier is permanently mechanically connected to the collecting unit. Therefore, the risk that the backflow barrier will become separated from the collecting unit and therefore will not be able to exert the desired blocking action is ruled out. The feature “permanently mechanically connected” does not, of course, rule out that the backflow barrier will be torn off or separated from the closure in a different manner in case of a sufficiently high force. During the regular operation, such a high force does not, however, occur as a rule.

According to the present invention, the restoring force generator aims to transfer the backflow barrier into the blocking state or into a blocking state and to hold it in the blocking state. The backflow barrier may only be transferred from the blocking state into the passing state against the restoring force, which the restoring force generator exerts. The flow of secretions through the patient-side port into the collecting unit interior space brings about that the backflow barrier is transferred against the restoring force into the passing state or into a passing state. This transfer into the passing state occurs only when the flow of secretions has reached the patient-side port. If this flow of secretions dries up, for example, because the suctioning is completed, the backflow barrier closes the patient-side port automatically by transferring the backflow barrier into a blocking state. A suitable action for closing the patient-side port is not necessary. Hence, the risk that a person inadvertently does not trigger this necessary action and as a result the patient-side port is not closed is ruled out. Thanks to the restoring force generator, rather, the backflow barrier is automatically moved into the blocking state, when only a little secretions or no more secretions at all flow into the collecting unit interior space, especially regardless of the bag and regardless of the quantity of secretions in the bag.

Especially if the bag is expanded by the received secretions or if the bag is pressed from outside, the situation may occur that secretions in the collecting unit interior space pushes against the blocking body. Because the blocking body is fastened to a surface of the closure, which points (faces) towards the collecting unit interior space, this leads to the backflow barrier being held in the blocking state by the restoring force generator and additionally by the fluid. The blocking body is located between the collecting unit interior space and the patient-side port. Even if only a relatively small quantity of secretions are in the bag and the bag is expanded only to a relatively limited extent, the backflow barrier remains in the blocking state thanks to the restoring force generator. This increases the operating safety. In particular, the risk that a large quantity of secretions will be released from the collecting unit is reduced.

The present invention makes it possible to configure the backflow barrier as a purely passive mechanical component. In particular, no actuating drive and no actuation of an actuating drive and no energy supply for such an actuating drive are necessary. The backflow barrier is less sensitive to contamination than a valve. The backflow barrier may have a configuration that is considerably simpler than a nonreturn valve. As a result, the backflow barrier can be embodied as space-saving.

The backflow barrier in the blocking state reduces the flow of secretions out of the collecting unit interior space. It does not necessarily completely prevent this flow of secretions. This is in many cases not necessary. Therefore, the backflow barrier can be positioned at the closure with a greater mechanical tolerance.

According to the present invention, the backflow barrier can be transferred into a blocking state and into a passing state. It is possible that the backflow barrier can be transferred into one of a plurality of possible different passing states, for example, in different possible positions in relation to the closure of the collecting unit.

According to the present invention, the blocking body of the backflow barrier is fastened to a top side of the closure, which faces towards the collecting unit interior space. As a result, the closure protects the blocking body against mechanical damage. In particular, the risk that a contamination leads to the blocking body not being sufficiently able to be freely moved and therefore the backflow barrier does not completely reach the blocking state and hence does not sufficiently prevent or even does not at all prevent a flow of secretions out of the collecting unit interior space is reduced. The blocking body can be arranged such that in the blocking state it extends in a plane, which is at right angles or obliquely to the flow direction of the secretions. This embodiment leads with even greater certainty to an undesirable flow of secretions out of the collecting unit interior space being prevented. In case of a valve, a valve body cannot, as a rule, be arranged in this manner.

According to the present invention, the blocking body is fastened to a top side of the closure, which faces toward the collecting unit interior space. In one embodiment, the following situation occurs when the backflow barrier is in the blocking state: The blocking body is in contact with the closure. The blocking body covers the patient-side port from the inside and prevents or reduces the flow of secretions out of the collecting unit interior space. The terms “on the inside” and “on the outside” refer to the collecting unit interior space.

Secretions typically flow through the patient-side fluid connection and through the patient-side port into the collecting unit interior space and hereby flows in a flow direction through the patient-side port. In one embodiment, the collection device is configured such that when the backflow barrier is in the blocking state, the following situation occurs: The blocking body extends in a plane, which is at right angles or at least obliquely to the flow direction of secretions through the patient-side port. As a result, secretions are prevented with relatively great certainty from escaping from the collecting unit interior through the patient-side port again, on the one hand, because secretions in the collecting unit interior push at right angles or obliquely onto the blocking body in this plane. It is ensured, on the other hand, with relatively great certainty that the secretions can flow into the collecting unit interior space through the patient-side port and no backup occurs.

The expansion of the blocking body in this plane in any direction is preferably greater than the expansion of the patient-side port. As a result, the blocking body completely blocks the patient-side port when the backflow barrier is in the blocking state. A distance preferably occurs between the blocking body—or at least an area of the blocking body—and the closure when the backflow barrier is in the passing state. Because of this distance, secretions can flow through the patient-side port into the collecting unit.

According to the present invention, the collecting unit encloses a collecting unit interior space, in which the suctioned secretions are received. The collecting unit preferably encloses this collecting unit interior space fully, up to at least one opening, through which the patient-side fluid connection is led and which is blocked in the blocking state by means of the backflow barrier according to the present invention, and optionally an opening, by means of which a vacuum is generated in the collecting unit interior space. In a preferred embodiment, the patient-side port is located entirely outside the collecting unit interior space, i.e., on an outer side of a wall of the collecting unit. The backflow barrier is located according to the present invention on an inner side of the collecting unit, which inner side points (faces) towards the collecting unit interior space, i.e., on an inner side of the wall. It is not accessible from outside during the suctioning.

This embodiment reduces the risk that the backflow barrier will unintentionally be opened due to a mechanical action from outside. In addition, this embodiment leads in many cases to the collecting unit, for example, an expanded bag of the collecting unit, pushing received secretions from the inside against the backflow barrier and as a result pushing the backflow barrier against the closure of the collecting unit. This action reinforces the action of the restoring force generator and further reduces the risk that the backflow barrier will be unintentionally moved out of the blocking state and that a greater quantity of secretions will be able to be discharged. This desired action is brought about without an additional adjusting element and without a special action, i.e., all by itself.

The following situation preferably occurs if the backflow barrier is in the passing state: The blocking body protrudes into the collecting unit interior space. As a result, a distance occurs between the closure and the blocking body. Secretions may flow through the space between the closure and the blocking body into the collecting room interior space. This space is often greater than a free space in a nonreturn valve, so that the backflow barrier opposes the secretions stream with less mechanical or pneumatic resistance.

According to the present invention, the blocking body is fastened to a surface of the closure, which surface points (faces) towards the collecting unit interior space. The blocking body is preferably fastened flush to this surface, especially with a distance to the opening, through which fluid (secretions) flow into the collecting unit interior space. As a result, the blocking body may be moved sufficiently and as a result selectively establish the passing state or the blocking state.

A stream of secretions into the collecting unit interior space transfers the backflow barrier into the passing state, especially against the restoring force of the restoring force generator, according to the present invention. In one embodiment, the blocking body engages with the flow of secretions, which flow into the collecting unit interior space. The engaging blocking body changes the direction of the secretions stream. The engaging blocking body thus at least partially deflects the secretions stream.

This embodiment especially reduces a foaming up of secretions in the collecting unit interior space. Such an intense foaming up is often undesirable. In addition, this deflection in many cases reduces the kinetic energy, with which the secretions stream impinges on an inner side of the collecting unit, for example, on an inner wall of a disposable bag of the collecting unit, and thus reduces the risk that the collecting unit will be mechanically damaged.

Different embodiments of the backflow barrier as a purely mechanical component are possible.

In one embodiment, the blocking body comprises a closing element, especially a membrane or a plate made of an elastic material, wherein the closing element can be deformed reversibly. The blocking body is preferably configured as such a closing element. The closing element is located in a blocking position in a non-deformed state, and it is located in a passing position in a deformed state. The closing element in the blocking position brings about the blocking state, the closing element in the flow position brings about the passing state or a passing state. If the closing element is deformed and as a result is brought into the flow position, then the deformed closing element exerts a restoring force, which aims to cancel the deformation of the closing element and as a result to again bring the closing element into the blocking position. The closing element itself thus applies the restoring force and at the same time acts as the restoring force generator. A separate element, which applies the restoring force, is not necessary.

This embodiment leads to a mechanical component which is especially simple. Only the reversibly deformable closing element as well as a suitable fastening of the closing element to the surface of the closure, which surface points (faces) towards the collecting unit interior space, are needed. Thanks to this embodiment, it is possible, but not necessary, to provide a joint, which mechanically connects the closing element to the collecting unit in a pivotable manner. The risk of malfunction is reduced. The collecting unit is often disposed of after a single use, so that the reversibly deformable closing element has to apply the restoring force only once after the suctioning.

In another embodiment, the locking body comprises a flap as well as preferably a joint, by means of which the flap is mechanically connected to the collecting unit. The blocking body is preferably configured as such a flap. The flap, which is preferably a rigid plate, can be moved back and forth, especially pivoted, between a blocking position and a passing position in relation to the collecting unit. The flap in the blocking position brings about the blocking state. The flap in the passing position brings about the passing state. The restoring force generator aims to move the flap into the blocking position and to hold it in this position. The restoring force generator preferably comprises at least one spring which is supported at the closure of the collecting unit and is connected to the flap in an articulated manner, and is especially connected in an articulated manner.

In one preferred embodiment, the volume of the collecting unit can be changed. For example, the collecting unit comprises an expandable bag, which is capable of receiving suctioned secretions. By contrast, the closure is preferably rigid. If the volume of the collecting unit is increased, a restoring force is brought about. This restoring force aims to reduce the volume of the collecting unit again. For example, an expanded bag aims to contract again. This restoring force aims to discharge the secretions out of the collecting unit.

According to the present invention, the blocking body is fastened to a surface of the closure, which surface faces towards the collecting unit interior space. The following state is preferably achieved when the backflow barrier is in the blocking state: The blocking body is expanded into a plane, which is at right angles to the flow direction of secretions. The restoring force, which is exerted by the collecting unit itself on the fluid in the collecting unit, aims to move the backflow barrier into the blocking state and to hold it in the blocking state. This restoring force of the collecting unit thus reinforces the restoring force from the restoring force generator. This embodiment thus further reduces the risk that a larger quantity of secretions will unintentionally be discharged out of the collecting unit.

The collecting unit comprises a bag, which is preferably expandable, as well as a closure, especially a cover, according to the present invention. The closure has a higher stiffness than the bag. These two components, i.e., the closure and the cover, can be manufactured separately from one another, preferably be made of different materials, and be connected to one another before a use. The bag is preferably expanded in order to connect it to the closure and the restoring force due to the expansion holds the bag at the closure, for example, at a circumferential ring of the closure, which ring faces inwards. Of course, the bag may additionally be connected in substance to the closure. The closure is located between the collecting unit interior space and the patient-side port. The bag is preferably connected permanently to the cover. The cover can preferably be inserted into a container made of a rigid material, and the bag protrudes from above into this container.

For example, the patient-side port protrudes outwards over the closure, so that a hose can be placed on and fastened to this patient-side port. Or the closure comprises a threaded section, into which a corresponding threaded section of a hose can be screwed.

In one embodiment, the collection device comprises a cap. The patient-side port can be closed by means of this cap. For example, the cap is placed onto the patient-side port from the outside. The present invention can be used in conjunction with such a cap, but spares the need to provide such a cap.

In conjunction with the backflow barrier in the blocking state, this cap reduces the risk that after the end of the suctioning secretions will be discharged out of the collecting unit. Thanks to the backflow barrier according to the present invention, such a cap is not necessary, however, in order to prevent this undesirable discharge. If the backflow barrier is used in conjunction with such a cap and the backflow barrier in the blocking state does not completely prevent the flow of secretions out of the collecting unit, but only reduces it, the cap only needs to be able to withstand a smaller stream of secretions than in a possible embodiment without a backflow barrier. The risk that the discharged secretions will unintentionally remove the cap from the patient-side port by itself is reduced.

The collection device according to the present invention is preferably a component of a collection system. This collection system comprises, furthermore, a container. The collection device is preferably used only once, namely in order to receive suctioned secretions from a patient once. The container is, by contrast, cleaned and used again after each use. The container carries the collection device according to the present invention and can, for example, be placed onto a support together with the collection device. The closure of the collecting unit is preferably placed onto the container, and the bag hangs into the container.

The collection device according to the present invention comprises a patient-side port, by means of which a patient-side fluid connection can be established between the collecting unit interior space and the patient-side coupling unit. Suctioned secretions flow into the collecting unit interior space through this patient-side fluid connection. The collection system comprises, furthermore, a device-side port. This device-side port may be arranged in the collecting unit, for example, in a closure of the collecting unit, or in the container. The device-side port makes it possible to establish a vacuum fluid connection between the collecting unit interior space and a vacuum source located outside the collection device. This vacuum source is preferably located outside the container. For example, this vacuum source generates a vacuum in the collecting unit, and the generated vacuum suctions secretions through the patient-side fluid connection into the collecting unit.

The collecting unit preferably comprises an expandable bag as well as a closure, for example, a cover, which is preferably placed onto the container. The closure and the container of the collection system together form a closed container made of a rigid material. This closed container protects the expandable bag against environmental effects. Secretions can preferably leave this container only through the patient-side port. However, the backflow barrier prevents this undesirable discharge of secretions or at least reduces it.

In one embodiment, the device-side port is fastened to the container. A channel in the closure of the collecting unit connects this device-side port to the collecting unit interior space in a fluid-tight manner. This embodiment avoids the need to connect a hose, which leads towards the vacuum source, directly to the closure. Rather, this hose can be connected to the device-side port at the container. This embodiment reduces the risk that the hose will unintentionally be detached or that a tear will occur in the closure.

This collection system is in one embodiment a component of a suctioning device. This suctioning device comprises, furthermore, a vacuum source and a patient-side coupling unit. A patient-side fluid connection is established or can be established between the patient-side coupling unit and the collecting unit interior space. This patient-side fluid connection leads through the patient-side port of the collection device. A vacuum fluid connection is established or can be established between the vacuum source and the collecting unit interior space. This vacuum fluid connection leads through the device-side port of the collection system.

The present invention will be described below on the basis of an exemplary embodiment. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a collection system, which comprises a collection device according to the present invention, a patient-side coupling unit and a vacuum source;

FIG. 2 is a sectional view showing a first embodiment of the backflow barrier according to the present invention in the form of a flexible membrane on the inner side of a cover;

FIG. 3 is a sectional view showing the backflow barrier from FIG. 2 with a modified cover; and

FIG. 4 is a sectional view showing a second embodiment of the backflow barrier according to the present invention in the form of a rigid cap or a spring on the inner side of the cover.

### DESCRIPTION OF PREFERRED EMBODIMENTS

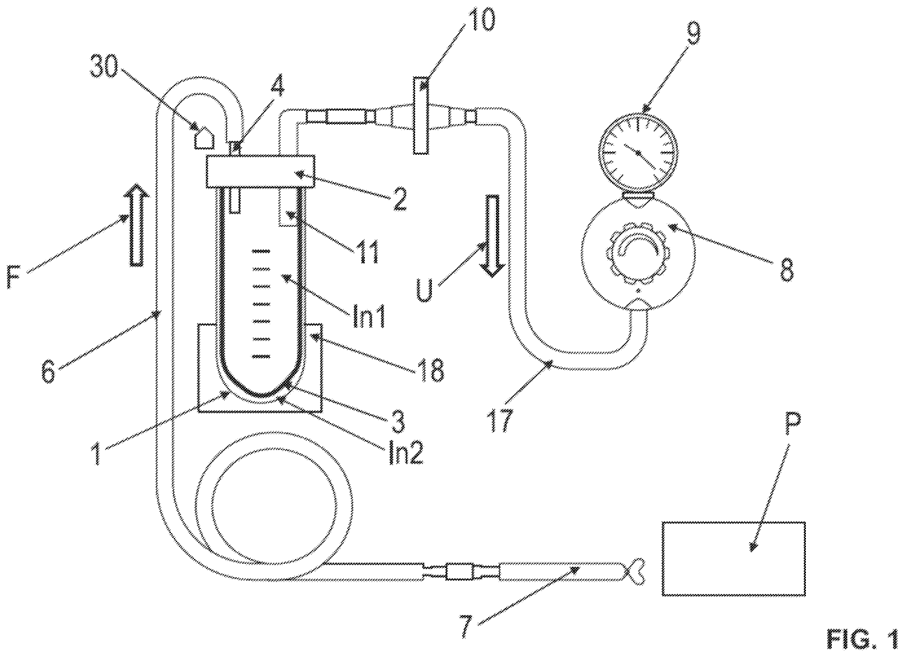

Referring to the drawings, FIG. 1 schematically shows a suctioning device, which comprises a collection system with a collection device according to the present invention. The suctioning device is connected from time to time to a patient P and is capable of suctioning and receiving secretions out of the patient P.

The collection device of the exemplary embodiment comprises
a cover 2, which is connected tightly, but detachably to the container 1,
an expandable bag 3, which hangs at the cover 2 and is connected to the cover 2 in a fluid-tight manner,
a hollow patient-side port 4, which is connected to the cover 2 permanently and in a fluid-tight manner and protrudes outwards, and
a cap 30, with which the patient-side port 4 can be closed.

The collection system comprises this collection device 2, 3, 4, 30 and furthermore
a container, which is permanently connected to a foot 18, which stands on the base or is placed detachably onto such a foot 18, wherein the expandable bag 3 protrudes into the container 1,
a suction hose 6, which can be placed onto the patient-side port or into the patient-side port 4 or be screwed into or inserted into same,
a cap 30, with which the patient-side port 4 can be closed when the suction hose 6 is pulled off, and
a device-side port 11 in a wall of the container 1.

The suctioning device comprises the collection system with the collection device according to the present invention and, moreover,
a catheter 7, which is connected to the suction hose 6 in a fluid-tight manner and is used at the patient P,
a vacuum source 8, which can be switched on and switched off,
a pressure difference measuring device 9, which measures and displays the vacuum, which the vacuum source 8 has generated,
a device-side hose 17, which connects the vacuum source 8 to the device-side port 11 in a fluid-tight manner, and
optionally a filter 10, which prevents drops of secretions from reaching the vacuum source 8.

The container 1 is manufactured from a solid plastic, is preferably transparent and is used a plurality of times. The container 1 is cleaned after each use.

The cover is manufactured from solid or flexible plastic. One or two grips are preferably arranged on the side of the cover 2 in order to be able to open and to carry the cover 2. The bag 3 is expandable, preferably transparent and preferably attached to the circumferential projection 19. Because the container 1 and the bag 3 are transparent, the fill level of secretions in the bag 3 can be seen from outside. The patient-side port 4 provides an opening Ö between the outer side of the cover 2 and the collecting unit interior space In1 in the bag 3.

The bag 3, the cover 2 and the patient-side port 4 belong to the collecting unit of the exemplary embodiment.

The cover 2 and the expandable bag 3 together enclose a collecting unit interior space In1. An interior space In2 is formed between the outer wall of the bag 3 and the inner wall of the container 1. The patient-side port 2 is in a fluid connection with the collecting unit interior space In1, wherein the opening Ö provided by the patient-side port 4 is led through the cover 2.

The suction hose 6 can be placed or pushed onto the patient-side port or into the patient-side port 4 or even screwed into the patient-side port 4. When the suction hose 6 is inserted or placed on or screwed in, the suction hose 6 encloses the patient-side port 4 in a fluid-tight manner. The cap 30 can then be placed onto the patient-side port 4 when the suction hose 6 is pulled off or removed from the patient-side port 4 in a different manner.

In one embodiment, the device-side port 11 is in fluid connection with the interior space In2. In a preferred embodiment, by contrast, the device-side port 11 is also in fluid connection with the collecting unit interior space In1. A channel, which establishes a fluid connection between the device-side port 11 and the collecting unit interior space In1 when the cover 2 is inserted, is led through the interior space of the cover 2.

The entire collecting unit 2,3, 4 is carried in a suitable packaging to the container and there the components are assembled to form the collection system in one embodiment. The cover 2, the patient-side port 4 and the bag 3 are used once, wherein the bag 3 receives suctioned secretions, and are subsequently removed from the container 1 and disposed of.

The suction device according to the exemplary embodiment is operated as described below, after the components are connected as just described and the catheter 7 is placed:
The vacuum source 8 is switched on.
The pressure difference measuring device 9 measures the vacuum generated in relation to the surrounding area and displays it. A control device, not shown, or even a monitoring person ensures that the generated vacuum is not greater than a predefined threshold, i.e., a maximum allowable vacuum is observed.
Air is suctioned out of the container 1 through the device-side port 11 and in a direction U through the device-side hose 17.
In one embodiment, the vacuum generated in the container 1 expands the bag 3. In another embodiment, air is suctioned out of the bag 3 through the channel in the cover 2. The bag 3 is expanded in both embodiments.
This vacuum in the bag 3 brings about that secretions are suctioned in the flow direction F out of the patient P through the catheter 7, through the suction hose 6, through the patient-side port 4 and through the channel in the cover 2 into the bag 3.
The suction hose 6 is removed from the patient-side port 4 as soon as enough secretions have been suctioned or the bag 3 is filled up to a predefined threshold. As a result, the suctioning is ended. The vacuum source 8 at first remains switched on in order to keep the bag 3 expanded.
The cap 30 is placed onto the patient-side port 4.
The vacuum source 8 is now switched off. It is not necessary to remove the device-side hose 17 from the device-side port 11 in a preferred embodiment.
The collecting unit with the filled bag 3, with the cover 3 and with the patient-side port 4 closed by means of the cap 30 is removed from the container 1 and disposed of.
The container 1 is cleaned and is available for a new use.

The vacuum that is generated in the container 1 expands the bag 3. As soon as the vacuum source 8 is switched off, the vacuum no longer opposes the aim of the bag 3 to contract again and as a result to discharge secretions. If it was forgotten to place the cap 30 onto the patient-side port 4, or if the cap is not placed on correctly, it may happen without the backflow barrier according to the present invention that a large quantity of secretions will abruptly be released from the bag 3 through the patient-side port 4 after switching off the vacuum source 8. This is undesirable. In addition, the collecting unit 2, 3, 4 may fall on the floor on the way to being disposed of, and a cap that is placed on incorrectly may fall off from the patient-side port 4.

The backflow barrier according to the present invention also prevents the undesirable event that secretions will be abruptly released from the bag 3 in these situations.

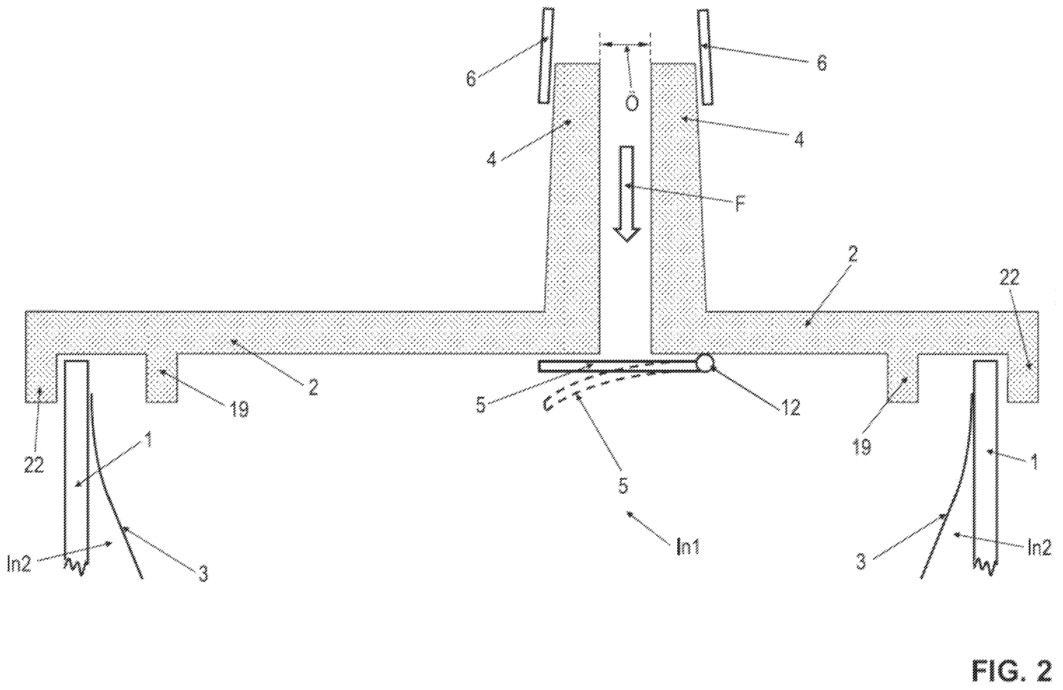

FIG. 2 through FIG. 4 schematically show in a sectional view two possible embodiments of a backflow barrier according to the present invention with a blocking body. Both embodiments provide a backflow barrier with a restoring force generator. The restoring force generator exerts a restoring force, which aims to bring the backflow barrier into a blocking state and to hold it in this state. The flow of secretions in the flow direction F into the bag 3 brings about that the backflow barrier is brought into a passing state against the restoring force. As soon as the flow of secretions dries up in the flow direction F, especially after switching off of the vacuum source 8, the restoring force automatically moves the backflow barrier into the blocking state. The backflow barrier prevents secretions from being abruptly released from the bag 3, even if the cap 30 is not placed on at all or is placed on incorrectly or if the collecting unit 2, 3, 4 falls onto the floor. If anything, secretions are only released from the patient-side port 4 in the form of drops thanks to the backflow barrier.

In FIG. 2 through FIG. 4, the following components are shown:
an upper area of the container 1,
the cover 2 with the ring-shaped circumferential section 19, which carries the bag 3,
optionally a circumferential sealing element 22 at the cover 2,
an upper part of the bag 3,
the patient-side port 4,
the opening Ö, which provides the patient-side port 4,
a part of the suction hose 6 connected to the patient-side port 4, which is placed onto the patient-side port 4, and
the flow direction F, in which suctioned secretions flow through the patient-side port 4 into the bag 3.

The cover 2 has the circumferential ring-shaped projection 19, which in one embodiment forms a ring-shaped groove, which extends around the upper edge of the container 1, and which is inserted into the container 1 in another embodiment. The optional sealing element 22 encloses the ring-shaped circumferential section 19 and has a distance to the circumferential section 19.

In all three embodiments, the backflow barrier is exclusively embodied with passive mechanical elements, i.e., without an actuation being necessary and without an adjusting element being needed. The elements of the backflow barrier are small, easy to manufacture and to assemble and can be disposed of together with the rest of the collecting unit 2, 3, 4, without a separate disposal of the backflow barrier being necessary.

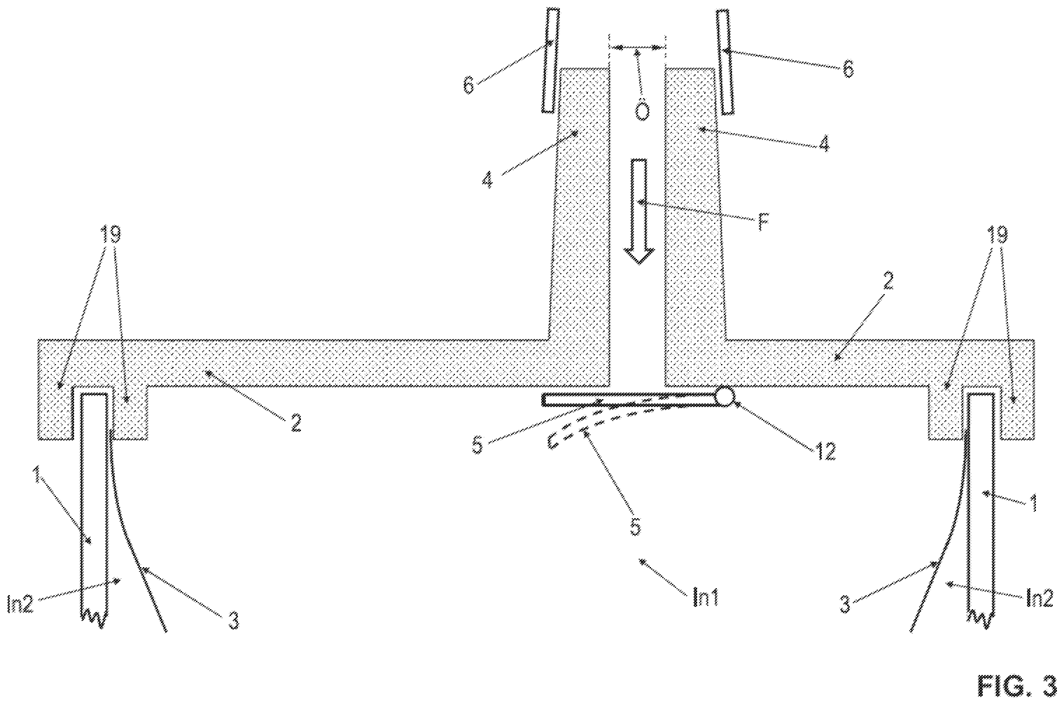

The backflow barrier of FIG. 2 and FIG. 3 comprises a reversibly deformable membrane 5, which is fastened to the cover 2 on the inside via a connection 12. The membrane 5 thus faces towards the interior space In1 of the collecting unit 2, 3, 4. The connection 12 is located on the inside at the cover 2 with a distance to the opening Ö. As soon as the bag 3 is fastened to the cover 2, the membrane 5 is not accessible from the outside.

The non-deformed membrane 5 (solid line) is flatly in contact with the cover 2 and brings about the blocking state. If after switching off the vacuum source 8, the bag 3 contracts and presses secretions in the direction of the cover 2, then the membrane is also pressed against the cover 2 and closes the opening Ö from the inside, especially completely or at least such that at most a few drops can be released from the patient-side port 4, i.e., the membrane 5 is held in the blocking state. In this embodiment, the membrane 5 itself applies the restoring force or at least a part of the restoring force.

The flow of secretions in the flow direction F brings about that the membrane 5 is deformed against the restoring force (broken line) and the opening Ö is no longer blocked, but lets secretions through. The membrane 5 engages with the flow of secretions, which is directed into the bag 3, and deflects the secretions stream. As a result, the risk that the secretions in the bag 3 will foam up, which is undesirable, is reduced.

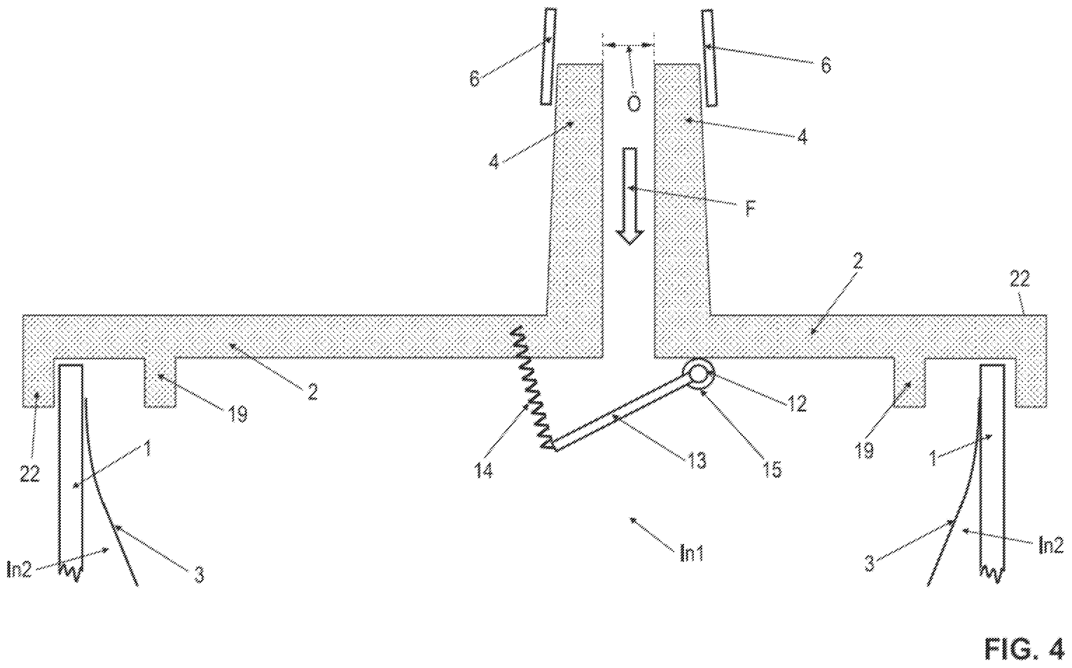

In the embodiment of FIG. 4, the flap 13 is fastened on the inside to the cover 2, especially to an axis 12 and such that the flap 13 can be rotated about the axis 12 in relation to the cover 2. The flap 13 is preferably configured as a rigid element. FIG. 4 shows the flap 13 in the passing state. A tension spring 14 is supported at the cover 2 and aims to pull the flap 13 towards the cover 2 and as a result to bring the flap 13 into the blocking state. An optional coil spring 15 is arranged around the axis 12 and likewise aims to bring the flap 15 into the blocking state. In turn, the contracting bag 3 brings about that pressed-out secretions press the flap 13 against the cover 2 and as a result closes the opening Ö.

While specific embodiments of the invention have been shown and described in detail to illustrate the application of the principles of the invention, it will be understood that the invention may be embodied otherwise without departing from such principles.

### LIST OF REFERENCE NUMBERS

1 Container; it accommodates the bag 3; it carries the device-side port 11; it belongs to the collection system
2 Cover for the container 1; it carries the patient-side port 4; it belongs to the collecting unit
3 Expandable bag, it receives secretions from the patient; it is held by the cover 2 in the interior space of the container 1; it belongs to the collecting unit
4 Hollow patient-side port; it is connected permanently to the cover 2; it is closed from the inside by the backflow barrier 5, 13, 16; it can be closed from the outside by the cap 30
5 Reversibly deformable closing element (membrane); it is attached to the cover 2; it belongs to the backflow barrier
6 Suction hose; it is connected to the patient-side port 4; it belongs to the patient-side coupling unit
7 Catheter; it is connected to the suction hose 6; it belongs to the patient-side coupling unit
8 Vacuum source; it comprises a pump; it is connected via the device-side hose 17 to the device-side port 11
9 Pressure difference measuring device, which measures and displays the vacuum generated by the vacuum source 8
10 Filter between the device-side port 11 and the vacuum source 8; it is arranged in the device-side hose 17
11 Device-side port; it is arranged at the container 1; it is connected to the vacuum source 8 via the device-side hose 17; it is in fluid connection with the bag 3
12 Mechanical connection between the backflow barrier 5, 13 and the cover 2
13 Rigid flap; it is pulled or pushed by the springs 14, 15 into the blocking state; it belongs to the backflow barrier
14 Tension spring; it is supported at the cover 2; it pulls the flap 13 into the blocking state
15 Coil spring; it rotates and pushes the flap 13 into the blocking state
17 Device-side hose; it connects the device-side port 11 to the vacuum source 8
18 Foot, on which the container 1 stands
19 Circumferential projection on the inside at the cover 2; it carries the bag 3; it is connected to the container 1
22 Circumferential sealing element at the cover 2; it encloses the circumferential projection 19
30 Cap for closing the patient-side port 4
F Flow direction of secretions through the suction hose 6
In1 Interior space in the bag 3; it is filled by suctioned secretions
In2 Space between the bag 3 and the inner wall of the container 1
Ö Opening, which connects the outer side of the cover 2 to the bag interior space In1; it can be closed by the backflow barrier 5, 13, 16
U Flow direction of air through the device-side hose 17

1. A collection device for receiving secretions suctioned out of a patient, the collection device comprising:
a collecting unit comprising a closure and a bag, the collecting unit enclosing a collecting unit interior space, the collecting unit being configured to receive secretions in the collecting unit interior space;
a patient-side port; and
a backflow barrier for the patient-side port, wherein the backflow barrier comprises a blocking body and a restoring force generator and is configured to be transferred into a passing state and into a blocking state, wherein the patient-side port is configured to enable a patient-side fluid connection between a patient-side coupling unit located outside the collecting unit and the collecting unit interior space, wherein the blocking body is fastened to a surface of the closure, which surface faces the collecting unit interior space, wherein the backflow barrier, in the blocking state, at least partially closes the patient-side port and at least reduces or inhibits a flow of secretions out of the collecting unit interior space into the patient-side port, wherein the restoring force generator exerts a restoring force on the blocking body which acts for transferring the backflow barrier into the blocking state and for keeping the backflow barrier in the blocking state, and wherein the backflow barrier is arranged such that a flow of secretions through the patient-side port into the collecting unit interior space brings about a transfer of the backflow barrier, against the restoring force, into the passing state.

2. A collection device in accordance with claim 1, wherein when the backflow barrier is in the blocking state, the blocking body is in contact with the closure and covers the patient-side port from an inside.

3. A collection device in accordance with claim 2, wherein with the backflow barrier in the blocking state, the blocking body extends in a plane, which is at right angles or is arranged obliquely to a flow direction of secretions through the patient-side port, and with the backflow barrier in the passing state, the blocking body protrudes into the collecting unit interior space.

4. A collection device in accordance with claim 1,
wherein an opening is formed in the closure through which the patient-side port is in a fluid connection with the collecting unit interior space,
wherein the blocking body is fastened to a surface of the closure at a spaced location from the opening.

5. A collection device in accordance with claim 1, wherein with the backflow barrier being in the passing state, the blocking body engages with a stream of secretions, which flow into the collecting unit interior space, and changes a direction of the stream of secretions.

6. A collection device in accordance with claim 1,
wherein the blocking body is configured as a reversibly deformable closing element,
wherein the closing element being in a non-deformed state is in a blocking position, which brings about the blocking state, and being in a deformed state is in a passing position, which brings about the passing state,
wherein the closing element in the blocking position is in contact with the closure, and
wherein the closing element in case of a deformation exerts a restoring force, acting against the deformation and thereby acting to bring the closing element into the blocking position.

7. A collection device in accordance with claim 1,
wherein the blocking body is configured as a rigid flap, and the restoring force generator is mechanically connected to the closure and to the flap,
wherein the flap is movable back and forth between a passing position, which brings about the passing state, and a blocked blocking position, which brings about the blocking state in relation to the closure, and
wherein the restoring force generator acts to move the flap into the blocked blocking position and to hold the flap in the blocked blocking position.

8. A collection device in accordance with claim 1,
wherein the bag is expandable,
wherein an expansion of the bag brings about a restoring force, which acts to reduce the volume of the bag,
wherein the collection device is configured such that the restoring force, which is brought about by an expansion of the bag, brings about secretions being in the collecting unit interior space being pushed against the blocking body and transferring the backflow barrier into the blocking state and keeping the backflow barrier in the blocking state.

9. (canceled)

10. A system comprising:
a collection device comprising:
a collecting unit comprising a closure and a bag, the collecting unit enclosing a collecting unit interior space, the collecting unit being configured to receive secretions in the collecting unit interior space;
a patient-side port configured to establish a patient-side fluid connection between a patient-side coupling unit located outside the collecting unit and the collecting unit interior space; and
a backflow barrier for the patient-side port, the backflow barrier comprising a blocking body and a restoring force generator and being configured to be transferred into a passing state and into a blocking state, the blocking body being fastened to a surface of the closure, which surface is directed towards the collecting unit interior space, in the blocking state, the blocking body at least partially closing the patient-side port to reduce or inhibit a flow of secretions out of the collecting unit interior space into the patient-side port, the restoring force generator exerting a restoring force on the blocking body which acts for transferring the backflow barrier into the blocking state and for keeping the backflow barrier in the blocking state, and wherein the backflow barrier is arranged such that a flow of secretions through the patient-side port into the collecting unit interior space brings about a transfer of the backflow barrier, against the restoring force, into the passing state;
a container; and
a device-side port, wherein the container accommodates and carries the collecting unit and wherein the device-side port is configured to enable a vacuum fluid connection to be established between the collecting unit interior space and a vacuum source outside the container.

11. A system according to claim 10, further comprising:
a vacuum source; and
a patient-side coupling unit, wherein the patient-side coupling unit is fluid-tightly connected to the patient-side port, wherein the vacuum source is fluid tightly connected to the device-side port, wherein a patient-side fluid connection is established or establishable between the patient-side coupling unit and the collecting unit interior space and leads through the patient-side port and wherein a vacuum fluid connection is established or establishable between the vacuum source and the collecting unit interior space and wherein the vacuum fluid connection leads through the device-side port.

12. A process for receiving secretions suctioned out of a patient, the process comprising the steps of:
providing a collection device, which collection device comprises a collecting unit, a patient-side port and a backflow barrier for the patient-side port, wherein the collecting unit comprises a closure and a bag and encloses a collecting unit interior space, wherein the backflow barrier comprises a blocking body and a restoring force generator, wherein the blocking body is fastened to a surface of the closure, which surface is directed towards the collecting unit interior space;
exerting, with the restoring force generator a restoring force, which keeps the backflow barrier in a blocking state;
connecting the patient-side port to a patient-side coupling, so that a patient-side fluid connection is established between the patient-side coupling unit and the collecting unit interior space, wherein the patient-side fluid connection leads through the patient-side port;
bringing about a flow of secretions through the patient-side fluid connection into the collecting unit;
with the backflow barrier is transferred against the restoring force into a passing state by the brought-about flow of secretions;
terminating the flow of secretions into the collecting unit;
with the restoring force again transferring the backflow barrier into the blocking state; and
with the backflow barrier in the blocking state completely or at least partially closing the patient-side port and as a result, reducing or inhibiting compared with the passing state a flow of secretions out of the collecting unit interior space through the patient-side port.
